(19) United States
(12) Patent Application Publication
(10) Pub. No.: US 2026/0232199 A1
(43) Pub. Date: Aug. 13, 2026

(54) BIOMETRIC INFORMATION ACQUISITION APPARATUS AND NON-TRANSITORY COMPUTER-READABLE STORAGE MEDIUM STORING BIOMETRIC INFORMATION ACQUISITION PROGRAM

(71) Applicant: SEIKO EPSON CORPORATION, TOKYO (JP)

(72) Inventors: Akira IKEDA, Chino-shi (JP); Satoshi SHIMIZU, SHIOJIRI-SHI (JP)

(21) Appl. No.: 19/539,126

(22) Filed: Feb. 13, 2026

(30) Foreign Application Priority Data
Feb. 13, 2025 (JP) 2025-021361

(51) Int. Cl. A61B 5/00

(52) U.S. Cl. CPC A61B 5/0077

(57) ABSTRACT

A biometric information acquisition apparatus includes: an imaging unit configured to image a living body and generate a plurality of pieces of frame image data; a face recognition unit configured to identify a face image contained in the frame image data and specify a plurality of feature points in the face image; and a detection unit configured to detect a pulse wave signal of the living body, in which the detection unit is configured to select a tracking feature point from the plurality of feature points, track the tracking feature point contained in each of the plurality of pieces of frame image data, and acquire the pulse wave signal based on a tracking feature point detection light amount of the tracking feature point in each of the plurality of pieces of frame image data.

The present application is based on, and claims priority from JP Application Serial Number 2025-021361, filed Feb. 13, 2025, the disclosure of which is hereby incorporated by reference herein in its entirety.

### BACKGROUND

### 1. Technical Field

The present disclosure relates to a biometric information acquisition apparatus and a non-transitory computer-readable storage medium storing a biometric information acquisition program.

### 2. Related Art

There is a biometric information acquisition apparatus that measures biometric information such as a pulse wave and a blood pressure of a subject. The biometric information measurement apparatus disclosed in JP-A-2016-190022 is an example of a biometric information acquisition apparatus. The biometric information measurement apparatus extracts a region of interest set in a skin region of a subject based on a luminance value of a video signal. The biometric information measurement apparatus tracks coordinates of the region of interest for each frame constituting the video and acquires coordinates of a tracking result. The biometric information measurement apparatus extracts a green signal of the region of interest and calculates a pulse wave based on the green signal.

JP-A-2016-190022 is an example of the related art.

When the skin region or the region of interest is tracked for each frame, the same coordinates may not be detected due to a scale of an image, a positional relationship with a light source, or the like.

### SUMMARY

A biometric information acquisition apparatus according to the present disclosure includes: an imaging unit configured to image a living body and generate a plurality of pieces of frame image data; a face recognition unit configured to identify a face image contained in the frame image data and specify a plurality of feature points in the face image; and a detection unit configured to detect a pulse wave signal of the living body, in which the detection unit is configured to select a tracking feature point from the plurality of feature points, track the tracking feature point contained in each of the plurality of pieces of frame image data, and acquire the pulse wave signal based on a tracking feature point detection light amount of the tracking feature point in each of the plurality of pieces of frame image data.

A non-transitory computer-readable storage medium storing a biometric information acquisition program causes a computer, which is coupled to an imaging unit that images a living body and generates a plurality of pieces of frame image data, to execute the following processing of: identifying a face image contained in the frame image data; specifying a plurality of feature points in the face image; selecting a tracking feature point included in the plurality of feature points; tracking the tracking feature point contained in each of the plurality of pieces of frame image data; and acquiring a pulse wave signal based on a tracking feature point detection light amount of the tracking feature point in each of the plurality of pieces of frame image data.

### BRIEF DESCRIPTION OF THE DRAWINGS

FIG. 1 is a view showing a schematic configuration of a measurement apparatus.

FIG. 2 is a block diagram showing the configuration of the measurement apparatus.

FIG. 3 is a view showing a captured image including face mesh information.

FIG. 4 is a view showing a part of the face mesh information.

FIG. 5 is a diagram showing an example of tone detection values.

FIG. 6 is a diagram showing an example of mesh point related information.

FIG. 7 is a diagram showing an example of a control flow executed by the measurement apparatus.

FIG. 8 is a diagram showing n-th face feature point time series data.

FIG. 9 is a diagram showing an example of an analysis procedure for detecting a pulse wave signal.

FIG. 10 is a diagram showing an analysis result of a noise-removed signal.

FIG. 11 is a diagram showing an example of a control flow executed by the measurement apparatus.

FIG. 12 is a diagram showing an example of a control flow executed by the measurement apparatus.

FIG. 13 is a diagram showing an example of a control flow executed by the measurement apparatus.

FIG. 14 is a diagram showing an example of a control flow executed by the measurement apparatus.

### DESCRIPTION OF EMBODIMENTS

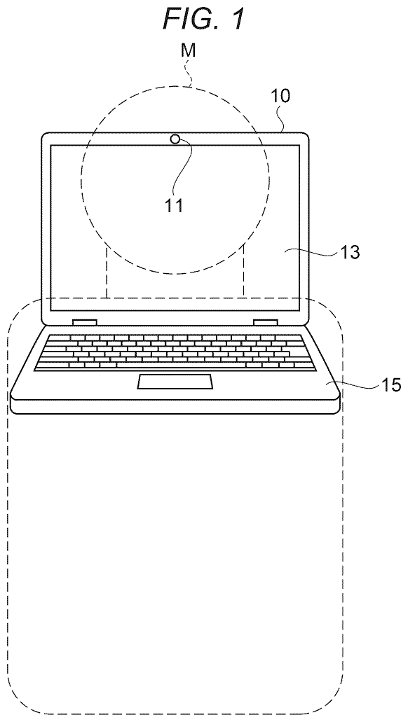

FIG. 1 shows a schematic configuration of a measurement apparatus 10. The measurement apparatus 10 corresponds to an example of a biometric information acquisition apparatus. The measurement apparatus 10 detects a pulse wave signal from a measurement operator M by using moving image data. The pulse wave signal is a signal indicating pulse waves of the measurement operator M. The measurement operator M corresponds to an example of a living body. The measurement apparatus 10 calculates biometric information of the measurement operator M based on a pulse wave signal. The biometric information includes pulse, pulse fluctuation, oxygen saturation concentration, blood pressure, and the like. The measurement apparatus 10 may evaluate sleep apnea syndrome or the like based on the biometric information. The measurement apparatus 10 displays the biometric information calculated based on the pulse wave signal.

The measurement apparatus 10 is implemented by an information processing apparatus such as a personal computer. The measurement apparatus 10 shown in FIG. 1 is a laptop computer, but is not limited thereto. The measurement apparatus 10 may be an apparatus having a function of capturing a moving image or an apparatus that can be coupled to a device for capturing a moving image. The measurement apparatus 10 is implemented by a desktop personal computer, a tablet terminal, a smartphone, or the like. The measurement apparatus 10 includes an imaging unit 11, a display unit 13, and an input unit 15. The measurement apparatus 10 may include a communication unit (not shown), or the like.

The imaging unit 11 images the measurement operator M by receiving reflected light, external light, or the like reflected by the measurement operator M or the like as detection light. The imaging unit 11 generates moving image data including the face of the measurement operator M. The moving image data is data for displaying a moving image, and includes a plurality of pieces of image data. The imaging unit 11 generates the plurality of pieces of image data. The image data corresponds to an example of frame image data. The image data includes a plurality of output values output in units of pixels. The image data is data used to cause the display unit 13 to display a captured image 100. The captured image 100 is a still image. The imaging unit 11 captures images at a predetermined frame rate to generate the moving image data. The imaging unit 11 corresponds to an example of an imager.

The imaging unit 11 is, for example, a camera including an optical element, an imaging element, and the like. The optical element condenses light on the imaging element. The imaging element converts detection light into an output value of an electric signal. The imaging element generates an output value for each of a plurality of pixels. An output value of each pixel indicates a light intensity of each pixel. The imaging element generates an output value of each pixel. The imaging element includes a charge coupled device (CCD), a complementary metal oxide semiconductor (CMOS), and the like. The output value includes tone values of a plurality of color light beams. The imaging unit 11 generates tone values of a plurality of color light beams for each pixel. The plurality of color light beams are, for example, red light, green light, and blue light. The red light, the green light, and the blue light have different wavelength bands. The red wavelength band, which is a wavelength band of the red light, is 600 nm to 800 nm. The green wavelength band, which is a wavelength band of the green light, is 520 nm to 550 nm. The blue wavelength band, which is a wavelength band of the blue light, is 430 nm to 490 nm. The imaging unit 11 may include infrared (IR) light or the like. The imaging unit 11 generates a red tone value that is a tone value of the red light, a green tone value that is a tone value of the green light, and a blue tone value that is a tone value of the blue light for each pixel. The image data includes an output value for each pixel including the red tone value, the green tone value, and the blue tone value. The image data includes luminance of each pixel.

The imaging unit 11 shown in FIG. 1 is a camera incorporated in the measurement apparatus 10, but is not limited thereto. The imaging unit 11 may be an external camera coupled to the measurement apparatus 10. The external camera is a near-infrared camera, a web camera, a smartphone camera, or the like.

The display unit 13 displays various kinds of information such as the captured image 100. The display unit 13 displays various kinds of biometric information based on a pulse wave signal. The display unit 13 may display a comment or the like based on the biometric information. The display unit 13 is implemented by a liquid crystal panel, an organic electro-luminescence (EL) panel, or the like. The display unit 13 may have a touch input function. When the touch input function is provided, the display unit 13 functions as the input unit 15. The display unit 13 shown in FIG. 1 is provided in the measurement apparatus 10, but is not limited thereto. The display unit 13 may be a display externally attached to the measurement apparatus 10.

The input unit 15 receives various input operations performed by the measurement operator M. The input unit 15 generates various input signals according to the input operations. The input unit 15 shown in FIG. 1 is a keyboard provided in the measurement apparatus 10, but is not limited thereto. The input unit 15 may be a mouse, a keyboard, a touch panel, a pen tablet, or the like coupled to the measurement apparatus 10.

The measurement operator M operates the measurement apparatus 10 at a position where the measurement operator faces the imaging unit 11 of the measurement apparatus 10. The measurement operator M operates the measurement apparatus 10 when the measurement apparatus 10 is caused to detect biometric information. The measurement operator M may operate the measurement apparatus 10 when performing a task such as document creation. The measurement apparatus 10 detects the biometric information related to the measurement operator M in the background when the measurement operator M is performing a task such as document creation. The measurement apparatus 10 can detect the biometric information related to the measurement operator M in a typical active state by detecting the biometric information in the background.

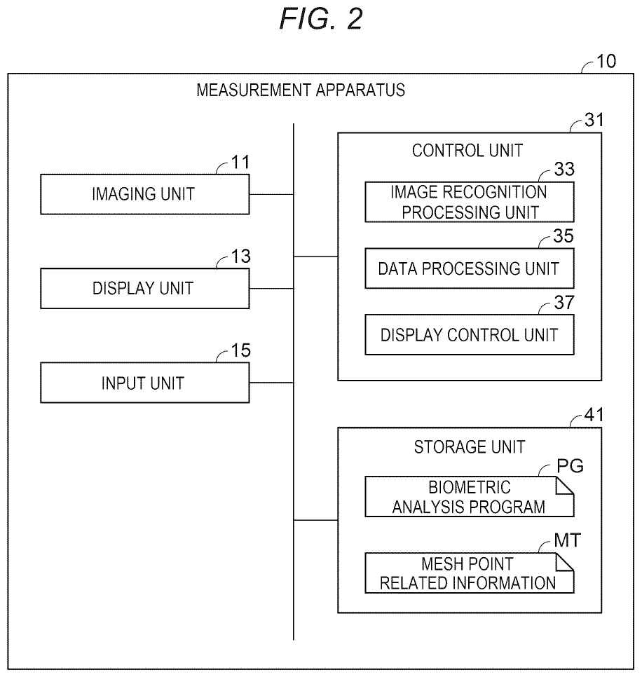

FIG. 2 is a block diagram showing the configuration of the measurement apparatus 10. The measurement apparatus 10 includes the imaging unit 11, the display unit 13, the input unit 15, a control unit 31, and a storage unit 41.

The imaging unit 11 transmits moving image data to the control unit 31. The imaging unit 11 transmits the moving image data to the control unit 31 at a predetermined timing. The imaging unit 11 may transmit image data contained in the moving image data to the control unit 31 at predetermined time intervals. The imaging unit 11 transmits image data implemented by output values including a red tone value, a green tone value, and a blue tone value for each pixel to the control unit 31. The imaging unit 11 may transmit the moving image data to the storage unit 41 and store the moving image data in the storage unit 41.

The display unit 13 displays various images under the control of the control unit 31. The display unit 13 receives display data from the control unit 31 and displays various images based on the display data. The display unit 13 may display a moving image captured by the imaging unit 11 based on the moving image data. The display unit 13 may display the captured image 100 based on image data contained in the moving image data.

The input unit 15 transmits an input signal to the control unit 31. The input unit 15 transmits the input signal to the control unit 31 to cause the control unit 31 to perform various types of control. For example, the input unit 15 transmits a display instruction signal for displaying biometric information to the control unit 31. The display instruction signal is an example of an input signal. Based on the display instruction signal, the control unit 31 generates biometric information display data for causing the display unit 13 to display biometric information based on a pulse wave signal. The control unit 31 transmits the biometric information display data to the display unit 13. The display unit 13 displays a screen including the biometric information based on the biometric information display data.

The control unit 31 is a controller that controls an operation of each unit. The control unit 31 is, for example, a processor including a central processing unit (CPU). The control unit 31 is implemented by one or more processors. The control unit 31 is communicatively coupled to the imaging unit 11, the display unit 13, and the like. The control unit 31 functions as an image recognition processing unit 33, a data processing unit 35, and a display control unit 37 by executing a biometric analysis program PG. The control unit 31 may function as a functional unit other than the image recognition processing unit 33, the data processing unit 35, and the display control unit 37 by executing the biometric analysis program PG. The control unit 31 corresponds to an example of a computer.

The image recognition processing unit 33 acquires the moving image data transmitted from the imaging unit 11. The image recognition processing unit 33 acquires a plurality of pieces of image data contained in the moving image data. The image recognition processing unit 33 identifies a face image contained in the image data. The image recognition processing unit 33 identifies a face image by executing face recognition processing on the image data. The face recognition processing is a processing of extracting a face image region 121 by detecting a face image feature point contained in the image data and matching the face image feature point with a face image database registered in advance. The face image database is a database that stores information related to a face image feature point used for face recognition. The face image feature point contained in the image data is, for example, a position and a contour of eye, nose, and mouth. The face image database is stored in the storage unit 41 in advance. When the measurement apparatus 10 is coupled to a server via a network, the face image database may be stored in the server in advance. The face image region 121 is a region where the face of the measurement operator M is displayed. The image recognition processing unit 33 identifies the face image region 121 by executing face recognition processing. The image recognition processing unit 33 corresponds to an example of a face recognition unit.

In the face recognition processing, for example, Face Mesh included in MediaPipe provided by Google (hereinafter, referred to as Face Mesh) is used. Face Mesh is a machine learning model that detects a key point of a face from an image. The image recognition processing unit 33 executes the face recognition processing using Face Mesh on each of a plurality of pieces of image data, and acquires the face image region 121 and face mesh information 131. The face mesh information 131 is represented by a plurality of key points contained in the face image region 121.

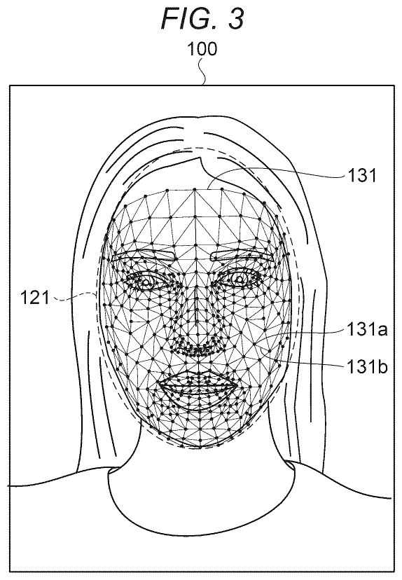

FIG. 3 shows the captured image 100 including the face mesh information 131. FIG. 3 shows an example of the face mesh information 131. The face mesh information 131 varies depending on the measurement operator M. FIG. 3 shows mesh points 131a and mesh lines 131b indicating the face mesh information 131.

The mesh point 131a is a point corresponding to a key point. The number of key points is, for example, 468. The image recognition processing unit 33 specifies a plurality of the mesh points 131a contained in the face image region 121. The image recognition processing unit 33 specifies a plurality of the mesh points 131a in each of a plurality of pieces of image data. The number of mesh points 131a specified in each of the plurality of pieces of image data is the same. A predetermined code is assigned to each of the plurality of mesh points 131a. The mesh points 131a to which the same code is assigned among the plurality of mesh points 131a contained in each of the plurality of pieces of image data correspond to the same position in the face image region 121. The mesh point 131a corresponds to an example of a feature point. The mesh line 131b is a line connecting two adjacent mesh points 131a.

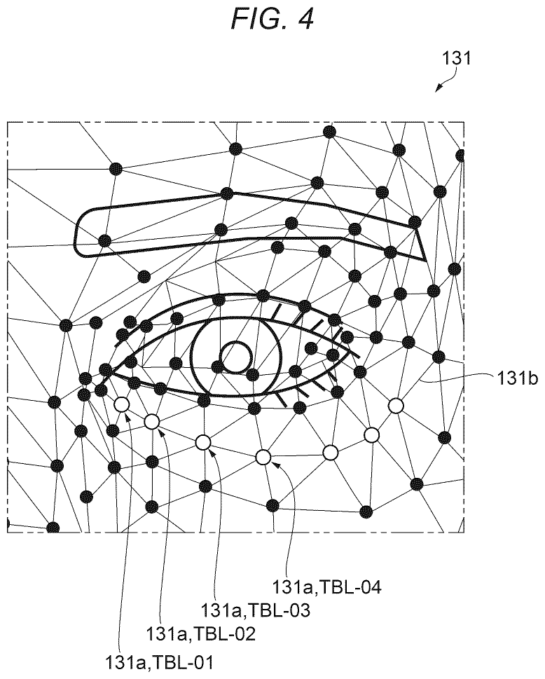

FIG. 4 shows a part of the face mesh information 131. FIG. 4 shows the face mesh information 131 near the left eye of the measurement operator M in an enlarged manner. FIG. 4 shows the plurality of mesh points 131a and a plurality of the mesh lines 131b. FIG. 4 shows code names of some mesh points 131a among the plurality of mesh points 131a.

FIG. 4 shows code names assigned to some mesh points 131a. The code names assigned to the mesh points 131a are, for example, a first code name TBL-01, a second code name TBL-02, a third code name TBL-03, and a fourth code name TBL-04. The code names shown in FIG. 4 indicate an eye bag of the left eye of the measurement operator M. A configuration of the code name can be set as appropriate. The image recognition processing unit 33 assigns a unique code name to each of the plurality of mesh points 131a.

The data processing unit 35 shown in FIG. 2 acquires the moving image data transmitted from the imaging unit 11. The data processing unit 35 acquires a plurality of pieces of image data contained in the moving image data. The data processing unit 35 acquires the red tone value, the green tone value, the blue tone value, luminance of a pixel, and the like for each pixel contained in the image data. The data processing unit 35 appropriately executes data processing such as correction processing on the red tone value, the green tone value, the blue tone value, and the like.

The data processing unit 35 detects a pulse wave signal of the measurement operator M. The data processing unit 35 analyzes biometric information based on the pulse wave signal. The data processing unit 35 corresponds to an example of a detection unit. The data processing unit 35 acquires, from the imaging unit 11, image data implemented by output values for each pixel including a red tone value, a green tone value, and a blue tone value. The data processing unit 35 acquires the face mesh information 131 from the image recognition processing unit 33. The data processing unit 35 associates the mesh points 131a contained in the face mesh information 131 with the output values of pixels. The data processing unit 35 associates the mesh points 131a with the output values of the pixels for each of the plurality of pieces of image data.

The data processing unit 35 tracks the predetermined mesh point 131a contained in the face mesh information 131 for each of the plurality of pieces of image data. The plurality of pieces of image data are generated in time series. The data processing unit 35 tracks a position of the predetermined mesh point 131a contained in each of the plurality of pieces of image data generated in time series by specifying a position of the predetermined mesh point 131a contained in each of the plurality of pieces of image data.

The data processing unit 35 may track one mesh point 131a among the plurality of mesh points 131a, or may track all of the plurality of mesh points 131a. The data processing unit 35 may track one or more mesh points 131a selected in advance based on a predetermined condition among the plurality of mesh points 131a. The selection of the mesh point 131a is appropriately set.

The data processing unit 35 associates the predetermined mesh point 131a with an output value of a pixel for each piece of image data. The data processing unit 35 acquires an output value associated with the predetermined mesh point 131a. The data processing unit 35 acquires an output value associated with the predetermined mesh point 131a for each piece of image data. The data processing unit 35 acquires a time series output value associated with the predetermined mesh point 131a as an output value group by acquiring an output value associated with the predetermined mesh point 131a for each piece of image data generated in time series.

The data processing unit 35 detects a pulse wave signal of the measurement operator M using the output value group associated with the predetermined mesh point 131a. The data processing unit 35 generates a detection value using a plurality of output values contained in the output value group. The detection value includes a tone detection value calculated using each tone value. The detection value may be the output value group associated with one mesh point 131a or a calculated value calculated based on a plurality of output value groups respectively associated with the plurality of mesh points 131a. The calculated value is, for example, an average value of the output values associated with the plurality of mesh points 131a for each piece of image data. The detection value is calculated for each piece of image data contained in the moving image data. The data processing unit 35 acquires a pulse wave signal based on the detection value of the predetermined mesh point 131a contained in each of the plurality of pieces of image data.

The data processing unit 35 detects the pulse wave signal using each tone detection value contained in the detection values. For example, the data processing unit 35 detects the pulse wave signal by using at least one of a red tone detection value Dr, a green tone detection value Dg, and a blue tone detection value Db. The red tone detection value Dr is calculated using the red tone value. The green tone detection value Dg is calculated using the green tone value. The blue tone detection value Db is calculated using the blue tone value. The pulse wave signal is detected based on the green tone detection value Dg. The pulse wave signal is detected based on a difference between the green tone detection value Dg and at least one of the red tone detection value Dr and the blue tone detection value Db.

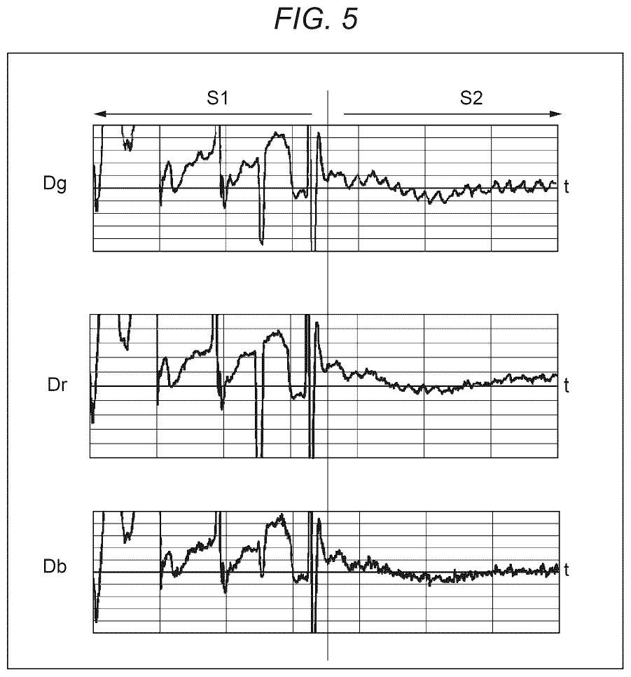

FIG. 5 shows an example of tone detection values. FIG. 5 shows the red tone detection value Dr, the green tone detection value Dg, and the blue tone detection value Db. FIG. 5 shows over-time changes in the red tone detection value Dr, the green tone detection value Dg, and the blue tone detection value Db in the form of waveform signals. FIG. 5 shows the red tone detection value Dr, the green tone detection value Dg, and the blue tone detection value Db in a body motion section S1, and the red tone detection value Dr, the green tone detection value Dg, and the blue tone detection value Db in a rest section S2. The body motion section S1 is a section in which a face moves or a facial expression changes. The rest section S2 is a section in which a face motion or a facial expression change is smaller than a predetermined change amount.

FIG. 5 shows the green tone detection value Dg. The green tone detection value Dg corresponds to a light amount value of the green light contained in the output value at one or more predetermined mesh points 131a. The green tone detection value Dg is contained in a detection value.

As shown in FIG. 5, in the body motion section S1, the green tone detection value Dg varies due to an influence of a body motion. A pulse wave signal contained in the green tone detection value Dg is less likely to be detected due to variation noises. In the rest section S2, the influence of variation noises on the green tone detection value Dg due to a body motion is reduced, and a pulse wave signal can be detected.

FIG. 5 shows the red tone detection value Dr. The red tone detection value Dr corresponds to a light amount value of the red light contained in the output value at one or more predetermined mesh points 131a. The red tone detection value Dr is contained in a detection value.

As shown in FIG. 5, in the body motion section S1, the red tone detection value Dr varies due to an influence of a body motion. A pulse wave signal contained in the red tone detection value Dr is less likely to be detected due to variation noises. In the rest section S2, the influence of the variation noises on the red tone detection value Dr due to a body motion is reduced, but an SN ratio is small, and thus it is less likely to detect a pulse wave signal.

FIG. 5 shows the blue tone detection value Db. The blue tone detection value Db corresponds to a light amount value of the blue light contained in the output value at one or more predetermined mesh points 131a. The blue tone detection value Db is contained in the detection values.

As shown in FIG. 5, in the body motion section S1, the blue tone detection value Db varies due to an influence of a body motion. A pulse wave signal contained in the blue tone detection value Db is less likely to be detected due to variation noises. In the rest section S2, the influence of the variation noises on the blue tone detection value Db due to a body motion is reduced, but an SN ratio is small, and thus it is less likely to detect a pulse wave signal.

The data processing unit 35 detects a pulse wave signal by using the red tone detection value Dr, the green tone detection value Dg, and the blue tone detection value Db shown in FIG. 5. The data processing unit 35 detects the pulse wave signal in an analysis procedure to be described later.

The data processing unit 35 shown in FIG. 2 calculates biometric information such as a pulse by calculating a cycle, an amplitude, and the like of the pulse wave signal. The data processing unit 35 transmits the biometric information including the pulse wave signal to the display control unit 37. The data processing unit 35 may store the biometric information and the like in the storage unit 41.

The display control unit 37 controls a display operation performed by the display unit 13. The display control unit 37 acquires the pulse wave signal and the biometric information from the data processing unit 35. The display control unit 37 generates biometric information display data including the biometric information. The display control unit 37 transmits the biometric information display data to the display unit 13. The display control unit 37 causes the display unit 13 to display the biometric information display data. The display control unit 37 can notify the measurement operator M of a detection result of the biometric information by causing the display unit 13 to display the biometric information display data.

The display control unit 37 may generate message data indicating an operation state of the biometric analysis program PG. The message data includes a start message, an execution message, an end message, and the like. The start message indicates that the detection of the biometric information is started. The execution message indicates that the biometric information is being detected. The end message indicates that the detection of the biometric information is ended. The display control unit 37 transmits the message data to the display unit 13. The display control unit 37 causes the display unit 13 to display the message data.

The storage unit 41 stores various programs, various kinds of data, and the like. The storage unit 41 stores the biometric analysis program PG and mesh point related information MT. The storage unit 41 stores a document creation program, a spreadsheet program, and the like. The storage unit 41 may store various kinds of data such as moving image data and biometric information. The storage unit 41 may store a face image database. The storage unit 41 is implemented by a semiconductor memory such as a random access memory (RAM) and a read only memory (ROM). The storage unit 41 may include a hard disk drive (HDD). The storage unit 41 may function as a work area for the control unit 31.

The biometric analysis program PG is a program for causing the measurement apparatus 10 to detect a pulse wave signal. The biometric analysis program PG is executed by the control unit 31. When the biometric analysis program PG is executed by the control unit 31, the control unit 31 functions as various functional units. The biometric analysis program PG is used to detect various kinds of biometric information based on the pulse wave signal. The biometric analysis program PG may be executed in the background when the control unit 31 executes a document creation program or the like. The biometric analysis program PG corresponds to an example of a biometric information acquisition program.

The mesh point related information MT is information related to the adjacent mesh points 131a among the plurality of mesh points 131a. The mesh point related information MT is generated when the image recognition processing unit 33 executes face recognition processing, and is stored in the storage unit 41. The mesh point related information MT is used when the data processing unit 35 selects one or more of the mesh points 131a from the plurality of mesh points 131a. The mesh point related information MT corresponds to an example of coupling information.

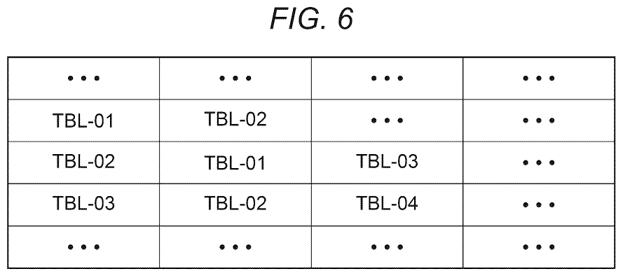

FIG. 6 shows an example of the mesh point related information MT. FIG. 6 shows the mesh point related information MT in a table format. FIG. 6 shows information related to the mesh points 131a of the first code name TBL-01, the second code name TBL-02, the third code name TBL-03, and the fourth code name TBL-04 shown in FIG. 4.

The mesh point related information MT shows the mesh point 131a and the mesh points 131a adjacent thereto. Two adjacent mesh points 131a are coupled by the mesh line 131b. The mesh point related information MT indicates one mesh point 131a and two or three or more mesh points 131a coupled by the mesh line 131b. For example, the mesh point related information MT indicates that the mesh point 131a of the first code name TBL-01 is located at a position adjacent to the mesh point 131a of the second code name TBL-02.

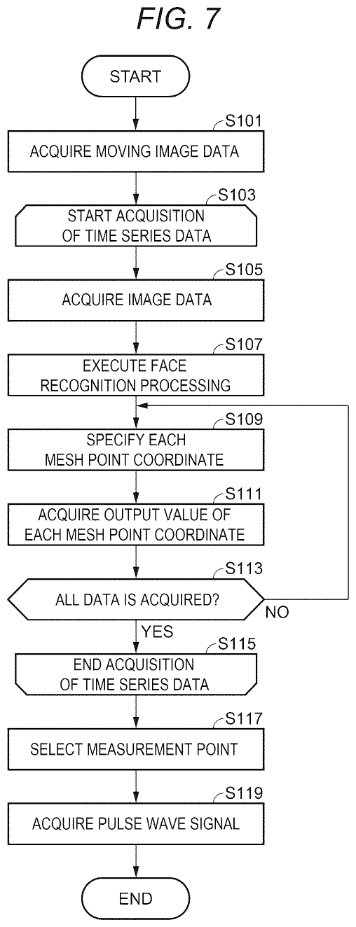

FIG. 7 shows an example of a control flow executed by the measurement apparatus 10. FIG. 7 shows a control flow for acquiring a pulse wave signal using an output value corresponding to the mesh point 131a. The control flow is executed by executing the biometric analysis program PG. FIG. 7 shows the control flow in a flowchart.

In step S101, the measurement apparatus 10 acquires moving image data. The measurement apparatus 10 causes the imaging unit 11 to generate the moving image data. The imaging unit 11 images the measurement operator M and generates the moving image data. The moving image data contains a plurality of pieces of image data. The plurality of pieces of image data are generated in time series. The imaging unit 11 generates the plurality of pieces of image data. When a frame rate at which the imaging unit 11 generates the moving image data is, for example, 30 frames per second (fps) and a measurement time is 8 seconds, the number of pieces of image data is 240. The imaging unit 11 transmits the moving image data to the control unit 31.

After acquiring the moving image data, the measurement apparatus 10 starts acquiring time series data in step S103. The data processing unit 35 of the control unit 31 acquires the moving image data transmitted from the imaging unit 11. The control unit 31 acquires the plurality of pieces of image data contained in the moving image data. The data processing unit 35 acquires the time series data of each mesh point 131a by executing the processing from step S103 to step S111. Details of the time series data will be described later.

In step S105, the measurement apparatus 10 acquires the image data. The image recognition processing unit 33 of the control unit 31 sequentially acquires the image data generated in time series. When the moving image data includes k pieces of image data, the image recognition processing unit 33 sequentially acquires the first image data to the k-th image data. k is a freely set integer. k is set according to a frame rate of the moving image data and a measurement time.

After acquiring the image data, the measurement apparatus 10 executes the face recognition processing in step S107. The image recognition processing unit 33 executes the face recognition processing on each of the plurality of pieces of image data. The image recognition processing unit 33 generates the face mesh information 131 contained in each piece of image data. The face mesh information 131 includes the plurality of the mesh points 131a and the plurality of the mesh lines 131b. The number of the mesh points 131a contained in each piece of image data is the same.

After executing the face recognition processing, the measurement apparatus 10 specifies mesh point coordinates of each mesh point 131a in step S109. The data processing unit 35 acquires the face mesh information 131 of each piece of image data. The data processing unit 35 specifies the mesh point coordinates of the plurality of mesh points 131a contained in each piece of image data. The mesh point coordinates are xy coordinates with a predetermined position in the image data as an origin. The face mesh information 131 includes N mesh points 131a including the n-th mesh point 131a. The n-th mesh point coordinates Cn, which are mesh point coordinates of the n-th mesh point 131a in one piece of image data, is represented by the following formula (1).

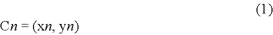

Here, n is any integer from 1 to N.

The n-th mesh point 131a contained in each of the plurality of pieces of image data indicates the same position in the face image region 121. The n-th mesh point coordinates Cn of each of the plurality of pieces of image data vary depending on a position and an orientation of the face of the measurement operator M in the image data. The control unit 31 can track the n-th mesh point 131a in time series by acquiring the n-th mesh point coordinates Cn contained in each piece of image data. The control unit 31 can track positions of all the mesh points 131a in time series by acquiring the n-th mesh point coordinates Cn of each of the plurality of pieces of image data.

After specifying the mesh point coordinates, the measurement apparatus 10 acquires output values of the mesh point coordinates in step S111. The data processing unit 35 acquires an output value of a pixel at a position matching the mesh point coordinates as an output value of the mesh point coordinates. The data processing unit 35 may acquire an output value of the mesh point coordinates based on an output value of a pixel at a position matching the mesh point coordinates and output values of peripheral pixels that are pixels in a predetermined region with respect to the pixel at the position matching the mesh point coordinates. The predetermined region is set in advance. When acquiring the output values of the peripheral pixels, the data processing unit 35 acquires, as the output value of the mesh point coordinates, for example, an average value or a total value of the output value of the pixel at the position matching the mesh point coordinates and the output values of the peripheral pixels. An n-th mesh point output value Bn, which is an output value of the n-th mesh point coordinates Cn in one piece of image data, is represented by the following formula (2).

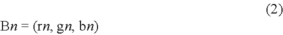

Here, rn is a red tone value contained in the output value of the n-th mesh point coordinates Cn, gn is a green tone value contained in the output value of the n-th mesh point coordinates Cn, and bn is a blue tone value contained in the output value of the n-th mesh point coordinates Cn.

The data processing unit 35 acquires n-th face feature point data Dn in the form of the following formula (3) by using the n-th mesh point coordinates Cn in the one piece of image data and the n-th mesh point output value Bn.

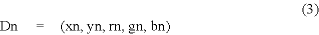

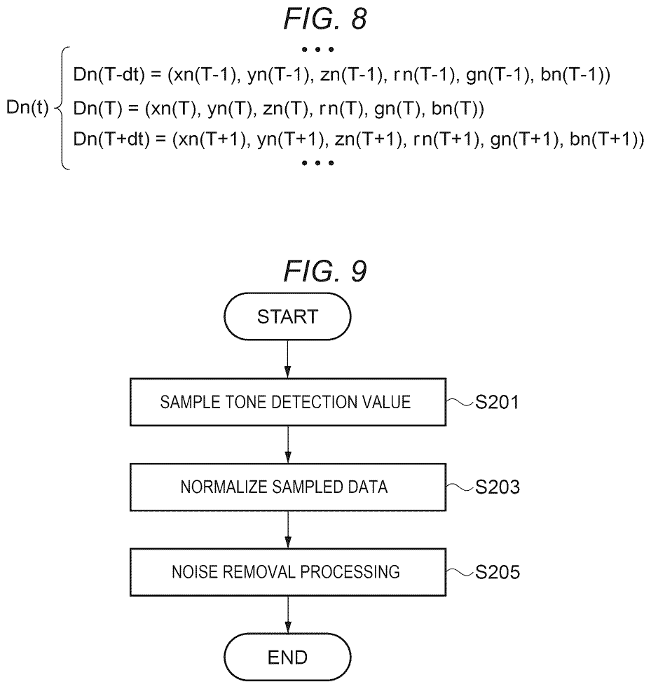

The data processing unit 35 acquires the n-th face feature point data Dn for each piece of image data. The data processing unit 35 acquires n-th face feature point time series data Dn(t) by acquiring the n-th face feature point data Dn for each piece of image data. The n-th face feature point time series data Dn(t) is an example of time series data of the n-th mesh point coordinates Cn. The n-th face feature point time series data Dn(t) includes T time n-th face feature point data Dn(T), T−dt time n-th face feature point data Dn(T−dt), and T+dt time n-th face feature point data Dn(T+dt) acquired at a time T at a frame rate of dt. Here, the time T is a freely set time from the start of measurement to the end of the measurement. The n-th face feature point time series data Dn(t) is shown in FIG. 8.

The data processing unit 35 acquires the n-th face feature point time series data Dn(t) shown in FIG. 8 by acquiring the n-th face feature point data Dn of the n-th mesh point coordinates Cn for each piece of image data. The n-th face feature point time series data Dn(t) is time series data of the n-th mesh point 131a tracked for each piece of image data.

After acquiring the output values of the mesh point coordinates, the measurement apparatus 10 determines whether the output values of all mesh point coordinates are acquired in step S113. The data processing unit 35 determines whether all the n-th face feature point time series data Dn(t) in which n is 1 to N are acquired. When it is determined that the data processing unit 35 acquires all the n-th face feature point time series data Dn(t), the measurement apparatus 10 proceeds the processing to step S115 (step S113: YES). When it is determined that the data processing unit 35 does not acquire all the n-th face feature point time series data Dn(t), the measurement apparatus 10 returns the processing to step S109 (step S113: NO). The measurement apparatus 10 continues to acquire the n-th face feature point time series data Dn(t).

In step S115, the measurement apparatus 10 ends the acquisition of the time series data. The data processing unit 35 acquires the n-th face feature point time series data Dn(t) in which n is 1 to N, and ends the acquisition of the time series data.

After acquiring the time series data, the measurement apparatus 10 selects a measurement point in step S117. The data processing unit 35 selects one mesh point 131a among the plurality of mesh points 131a as a tracking measurement point. The tracking measurement point is an example of a measurement point and corresponds to an example of a tracking feature point. The data processing unit 35 may select, as analysis measurement points, a plurality of mesh points 131a including the one mesh point 131a selected as the tracking measurement point. The data processing unit 35 may select, as the analysis measurement points, all the mesh points 131a including the one mesh point 131a selected as the tracking measurement point. The analysis measurement point is an example of a measurement point and corresponds to an example of a measurement feature point. A method of selecting a measurement point will be described later.

After selecting the measurement point, the measurement apparatus 10 acquires a pulse wave signal in step S119. The data processing unit 35 acquires a pulse wave signal using the n-th face feature point time series data Dn(t) of a measurement point. The n-th face feature point time series data Dn(t) includes an output value group associated with the predetermined mesh point 131a. The output value group is the n-th mesh point output value Bn contained in the n-th face feature point time series data Dn (t). The data processing unit 35 generates a detection value using the n-th mesh point output value Bn. The data processing unit 35 detects the red tone detection value Dr, the green tone detection value Dg, and the blue tone detection value Db shown in FIG. 5 using the n-th face feature point time series data Dn(t).

FIG. 9 shows an example of an analysis procedure for detecting the pulse wave signal. FIG. 9 is a flowchart showing the example of the analysis procedure. The analysis procedure shown in FIG. 9 is executed by the data processing unit 35. In the analysis procedure shown in FIG. 9, the pulse wave signal is detected by using the red tone detection value Dr, the green tone detection value Dg, and the blue tone detection value Db.

In step S201, the data processing unit 35 samples tone detection values at a predetermined time interval. The time interval and a sampling frequency are set as appropriate. The time interval is preferably a time containing one or more pulse waves. The time interval is, for example, 3 seconds to 10 seconds. The sampling frequency is, for example, 10 Hz or more and 50 Hz or less. The data processing unit 35 acquires sampled data by performing the sampling. The sampled data contains the sampled red tone detection values Dr, green tone detection values Dg, and blue tone detection values Db.

After performing the sampling, the data processing unit 35 normalizes the sampled data in step S203. The data processing unit 35 normalizes the green tone detection value Dg contained in the sampled data.

The data processing unit 35 calculates a green average value Gmean, which is an average value of a plurality of the green tone detection values Dg, and a green standard deviation value Gstd, which is a standard deviation value of a plurality of the green tone detection values Dg. The data processing unit 35 normalizes each green tone detection value Dg using the following formula (4).

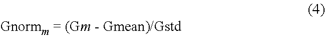

Here, m is any integer of 1 or more. Gm is the m-th green tone detection value Dg. Gnormₘ is a value obtained by normalizing the m-th green tone detection value Dg.

The data processing unit 35 normalizes a plurality of the red tone detection values Dr and a plurality of the blue tone detection values Db contained in the sampled data in a similar manner to the green tone detection value Dg. The data processing unit 35 calculates a red average value Rmean, which is an average value of the plurality of red tone detection values Dr, and a red standard deviation value Rstd, which is a standard deviation value of the plurality of red tone detection values Dr. The data processing unit 35 calculates a blue average value Bmean, which is an average value of a plurality of the blue tone detection values Db, and a blue standard deviation value Bstd, which is the standard deviation value of the plurality of blue tone detection values Db. The data processing unit 35 normalizes each of the red tone detection values Dr and each of the blue tone detection values Db by using the following formulas (5) and (6).

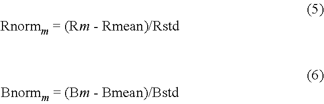

Here, m is any integer of 1 or more. Rm is the m-th red tone detection value Dr. Rnormₘ is a value obtained by normalizing the m-th red tone detection value Dr. Bm is the m-th blue tone detection value Db. Bnormₘ is a value obtained by normalizing the m-th blue tone detection value Db.

After normalizing the sampled data, the data processing unit 35 executes noise removal processing in step S205. The data processing unit 35 executes the noise removal processing by using the normalized green tone detection values Dg, the normalized red tone detection values Dr, and the normalized blue tone detection values Db. The data processing unit 35 executes the noise removal processing using the following formula (7) to generate a noise-removed signal S.

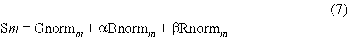

Here, m is any integer of 1 or more. Sm is an m-th noise-removed signal S. α is a first coefficient, and β is a second coefficient.

For example, α and β are each −0.5. When α and β are negative values, the data processing unit 35 detects the noise-removed signal S by subtracting the normalized red tone detection values Dr and the normalized blue tone detection values Db from the normalized green tone detection values Dg. At least one of α and β may be zero. When α=0 and β=−0.5, the data processing unit 35 detects the noise-removed signal S by calculating a difference between the green tone detection value Dg and the red tone detection value Dr. When α=−0.5 and β=0, the data processing unit 35 detects the noise-removed signal S by calculating a difference between the green tone detection value Dg and the blue tone detection value Db. The coefficients α and β are set as appropriate in accordance with a state of the noise removal.

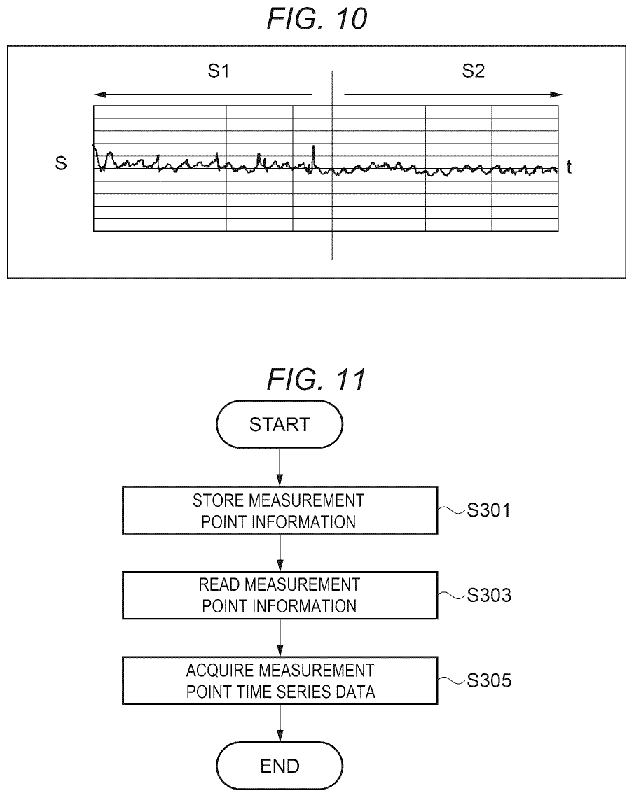

FIG. 10 shows an analysis result of the noise-removed signal S. FIG. 10 shows an analysis based on the red tone detection value Dr, the green tone detection value Dg, and the blue tone detection value Db shown in FIG. 5. FIG. 10 shows the noise-removed signal S when α=−0.5 and β=−0.5 are substituted into the formula (7). FIG. 10 shows the noise-removed signal S in the body motion section S1 and the rest section S2.

The noise-removed signal S corresponds to a pulse wave signal, as shown in FIG. 10. Noise components such as a body motion are removed from the noise-removed signal S. The data processing unit 35 detects the noise-removed signal S as a pulse wave signal. A signal waveform of the noise-removed signal S in the rest section S2 is detected more clearly than that of the green tone detection value Dg. The noise-removed signal S in the body motion section S1 is adjusted to a signal waveform corresponding to the pulse wave signal. By executing the noise removal processing, the data processing unit 35 can detect pulse wave signals in the body motion section S1 and the rest section S2.

The data processing unit 35 may calculate biometric information such as a pulse wave using the noise-removed signal S. The data processing unit 35 acquires the noise-removed signal S as a pulse wave signal. The data processing unit 35 calculates biometric information such as a pulse by calculating a cycle, an amplitude, and the like of the pulse wave signal. The data processing unit 35 transmits the biometric information including the pulse wave signal to the display control unit 37. The data processing unit 35 may store the biometric information and the like in the storage unit 41.

The data processing unit 35 acquires the pulse wave signal using one or any number of pieces of n-th face feature point time series data Dn(t) among N pieces of n-th face feature point time series data Dn(t) in which n is 1 to N.

FIG. 11 shows an example of a control flow executed by the measurement apparatus 10. FIG. 11 shows the control flow using measurement point information. FIG. 11 shows an example of the control flow executed in step S117 shown in FIG. 7. FIG. 11 shows a control flow for selecting one or more mesh points 131a as a measurement point based on the measurement point information and acquiring time series data of the measurement point. The measurement apparatus 10 acquires the pulse wave signal in step S119 shown in FIG. 7 using the time series data of the selected mesh point 131a.

In step S301, the measurement apparatus 10 stores the measurement point information. One mesh point 131a is selected in advance as a tracking measurement point from all the mesh points 131a. When a plurality of the mesh points 131a are selected from all the mesh points 131a, the plurality of mesh points 131a are selected as analysis measurement points. Any one of the plurality of analysis measurement points is set as a tracking measurement point. The tracking measurement point and the analysis measurement point are selected in advance by the measurement operator M, an administrator of the biometric analysis program PG, or the like. The measurement point information includes information related to the tracking measurement point or the analysis measurement point. The information related to the tracking measurement point is identification information for identifying the mesh point 131a which is a tracking measurement point. The information related to the analysis measurement point is identification information for identifying the mesh point 131a which is the analysis measurement point. The identification information is, for example, a code name shown in FIG. 4. The measurement point information is stored in the storage unit 41 before the control flow shown in FIG. 7 is executed.

In step S303, the measurement apparatus 10 reads the measurement point information. In step S115 of FIG. 7, after the acquisition of the time series data ends, the data processing unit 35 reads the measurement point information from the storage unit 41.

After reading the measurement point information, the measurement apparatus 10 acquires measurement point time series data in step S305. The data processing unit 35 reads identification information corresponding to the tracking measurement point or the analysis measurement point by using the measurement point information. The data processing unit 35 acquires time series data of the mesh point 131a identified by the identification information. The data processing unit 35 acquires time series data of the mesh points 131a, which are tracking measurement points, as tracking measurement point time series data. The tracking measurement point time series data includes an output value group of the tracking measurement points. The tracking measurement point time series data is an example of measurement point time series data, and corresponds to an example of a tracking feature point detection light amount. The data processing unit 35 acquires time series data of the mesh points 131a, which are analysis measurement points, as analysis measurement point time series data. The analysis measurement point time series data includes an output value group of the analysis measurement points. The analysis measurement point time series data is an example of measurement point time series data, and corresponds to an example of a measurement feature point detection light amount.

The data processing unit 35 acquires a pulse wave signal in step S119 shown in FIG. 7 by using the tracking measurement point time series data. The data processing unit 35 tracks the predetermined mesh point 131a and acquires the pulse wave signal using the output value group of the tracked mesh point 131a, thereby improving the measurement accuracy of the pulse wave signal.

The data processing unit 35 may acquire the pulse wave signal in step S119 shown in FIG. 7 by using the analysis measurement point time series data. The analysis measurement point time series data includes the tracking measurement point time series data. The data processing unit 35 acquires an output value group of analysis measurement points contained in the analysis measurement point time series data. The data processing unit 35 calculates an average mesh point output value Bave using the mesh point output value of the analysis measurement point for each piece of image data. The average mesh point output value Bave is represented by the following formula (8).

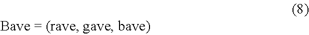

Here, rave is an average red tone value calculated based on an output value of an analysis measurement point, gave is an average green tone value calculated based on an output value of an analysis measurement point, and bave is an average blue tone value calculated based on an output value of an analysis measurement point.

The data processing unit 35 calculates average mesh point output value time series data Bave(t) using the average mesh point output value Bave. The average mesh point output value time series data Bave(t) is an example of the analysis measurement point time series data. The average mesh point output value time series data Bave(t) is represented by the following formula (9).

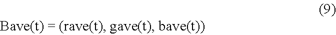

The data processing unit 35 acquires a pulse wave signal in step S119 shown in FIG. 7 using the average mesh point output value time series data Bave(t) which is the analysis measurement point time series data. The data processing unit 35 tracks the plurality of mesh points 131a and acquires the pulse wave signal using an output value group of the plurality of mesh points 131a, thereby improving measurement accuracy of the pulse wave signal.

The measurement apparatus 10 includes the imaging unit 11 that images the measurement operator M and generates a plurality of pieces of image data, the image recognition processing unit 33 that identifies a face image contained in the image data and specifies a plurality of the mesh points 131a in the face image, and the data processing unit 35 that detects a pulse wave signal of the measurement operator. The data processing unit 35 selects a tracking measurement point from the plurality of mesh points 131a, tracks the tracking measurement point contained in each of the plurality of pieces of image data, and acquires the pulse wave signal based on the tracking measurement point time series data of the tracking measurement point in each of the plurality of pieces of image data.

The data processing unit 35 tracks the predetermined mesh point 131a and acquires the pulse wave signal using the tracking measurement point time series data of the predetermined mesh point 131a, thereby improving the measurement accuracy of the pulse wave signal.

It is preferable that the data processing unit 35 selects a plurality of analysis measurement points including the tracking measurement point from the plurality of mesh points 131a, tracks the analysis measurement point contained in each of the plurality of pieces of image data, calculates the analysis measurement point time series data of analysis measurement points contained in each of the plurality of pieces of image data, and acquires the pulse wave signal using a plurality of pieces of the analysis measurement point time series data.

The data processing unit 35 tracks the plurality of mesh points 131a and acquires the pulse wave signal using the analysis measurement point time series data of the plurality of mesh points 131a, thereby further improving the measurement accuracy of the pulse wave signal.

The biometric analysis program PG causes the control unit 31, which is coupled to the imaging unit 11 that images the measurement operator M and generates a plurality of pieces of image data, to identify a face image contained in the image data, specify the plurality of mesh points 131a in the face image, select the tracking measurement point included in the plurality of mesh points 131a, track the tracking measurement point contained in each of the plurality of pieces of image data, and acquire the pulse wave signal based on the tracking measurement point time series data of the tracking measurement point in each of the plurality of pieces of image data.

The data processing unit 35 tracks the predetermined mesh point 131a and acquires the pulse wave signal using the tracking measurement point time series data of the predetermined mesh point 131a, thereby improving the measurement accuracy of the pulse wave signal.

When acquiring the pulse wave signal using the measurement point information, the measurement apparatus 10 may or may not acquire the time series data of all the mesh points 131a. The measurement apparatus 10 may specify one or more mesh points 131a for which time series data is acquired using the measurement point information, and acquire the time series data of the specified mesh point 131a. The measurement apparatus 10 acquires the pulse wave signal using the time series data of the specified mesh point 131a.

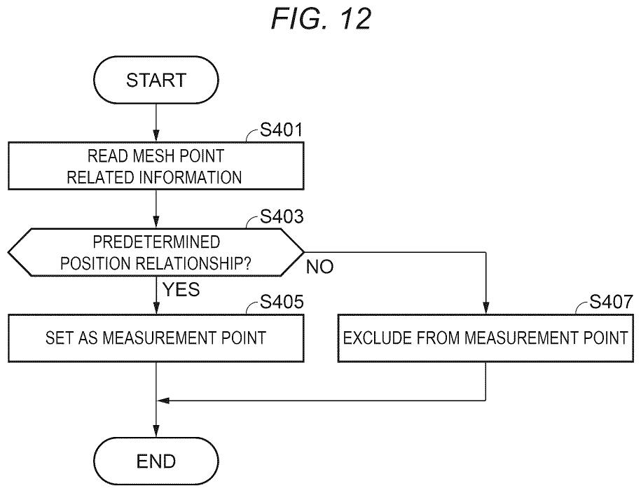

FIG. 12 shows an example of a control flow executed by the measurement apparatus 10. FIG. 12 shows a control flow for specifying a measurement point using the mesh point related information MT. FIG. 12 shows an example of the control flow executed in step S117 shown in FIG. 7. In FIG. 12, a measurement point is specified using mesh point coordinates contained in the time series data. The measurement apparatus 10 acquires the pulse wave signal in step S119 shown in FIG. 7 using the time series data of the specified measurement point.

In step S401, the measurement apparatus 10 reads the mesh point related information MT from the storage unit 41. For example, the data processing unit 35 reads the mesh point related information MT shown in FIG. 6. The mesh point related information MT includes information indicating the mesh points 131a adjacent to each other and coupled by the mesh line 131b.

After reading the mesh point related information MT, the measurement apparatus 10 determines whether mesh point coordinates of two adjacent mesh points 131a satisfy a predetermined positional relationship in step S403. The mesh point coordinates correspond to an example of coordinate information. Each mesh point 131a is set at a predetermined position in the face image region 121. When the face of the measurement operator M directly faces the imaging unit 11, the mesh point coordinates of two adjacent mesh points 131a in the image data satisfy a predetermined positional relationship. When the face of the measurement operator M changes from a direction facing the imaging unit 11 to a different direction, the mesh point coordinates of two adjacent mesh points 131a in the image data may not satisfy the predetermined positional relationship.

For example, when two mesh point coordinates are set in which mesh point coordinates of any one mesh point 131a are (x1, y1) and mesh point coordinates of the mesh point 131a adjacent to the right of the one mesh point 131a in the image data are (x2, y2), x1 and x2 satisfy a relationship of x1<x2. Here, an origin of the mesh point coordinates is set at a lower left of the image data. When the face of the measurement operator M faces the left, x1 and x2 may satisfy a relationship of x1≥x2.

The data processing unit 35 selects one mesh point 131a from the plurality of mesh points 131a. The data processing unit 35 specifies the adjacent mesh point 131a adjacent to the one mesh point 131a using the mesh point related information MT. The data processing unit 35 compares the mesh point coordinates of the one mesh point 131a with the mesh point coordinates of the adjacent mesh point 131a for each piece of image data. The data processing unit 35 determines whether the one mesh point 131a and the adjacent mesh point 131a satisfy a predetermined positional relationship. The data processing unit 35 sequentially selects all the mesh points 131a as the one mesh point 131a. The data processing unit 35 compares the mesh point coordinates of the mesh point 131a selected for each mesh point 131a with the mesh point coordinates of the adjacent mesh point 131a specified using the mesh point related information MT. The data processing unit 35 determines whether the selected mesh point 131a and the adjacent mesh point 131a satisfy a predetermined positional relationship. When the data processing unit 35 determines that the one mesh point 131a and the adjacent mesh point 131a satisfy the predetermined positional relationship, the measurement apparatus 10 proceeds the processing to step S405 (step S403: YES). When the data processing unit 35 determines that the one mesh point 131a and the adjacent mesh point 131a do not satisfy the predetermined positional relationship, the measurement apparatus 10 proceeds the processing to step S407 (step S403: NO).

In step S405, the measurement apparatus 10 sets the one mesh point 131a as a measurement point. The set measurement point is a tracking measurement point or an analysis measurement point. The data processing unit 35 selects a measurement point from the plurality of mesh points 131a by setting the one mesh point 131a as a measurement point. The data processing unit 35 acquires time series data of the mesh point 131a set as the measurement point. The data processing unit 35 acquires a pulse wave signal in step S119 shown in FIG. 7 using the acquired time series data.

In step S407, the measurement apparatus 10 excludes the one mesh point 131a from a measurement point. The data processing unit 35 does not use the time series data of the mesh point 131a excluded from the measurement point for acquisition of the pulse wave signal.

The data processing unit 35 preferably acquires the mesh point related information MT related to the adjacent mesh points 131a among the plurality of mesh points 131a.

The data processing unit 35 can confirm the adjacent mesh points 131a by acquiring the mesh point related information MT.

The data processing unit 35 preferably selects a measurement point using the mesh point related information MT.

The data processing unit 35 can easily select a measurement point by using the mesh point related information MT.

The data processing unit 35 preferably specifies mesh point coordinates of each of the plurality of mesh points 131a and selects an analysis measurement point based on the mesh point coordinates.

The measurement apparatus 10 can exclude the mesh point 131a that does not satisfy a predetermined positional relationship depending on an orientation of the face of the measurement operator M from a measurement point. The measurement apparatus 10 can improve the detection accuracy of the pulse wave signal.

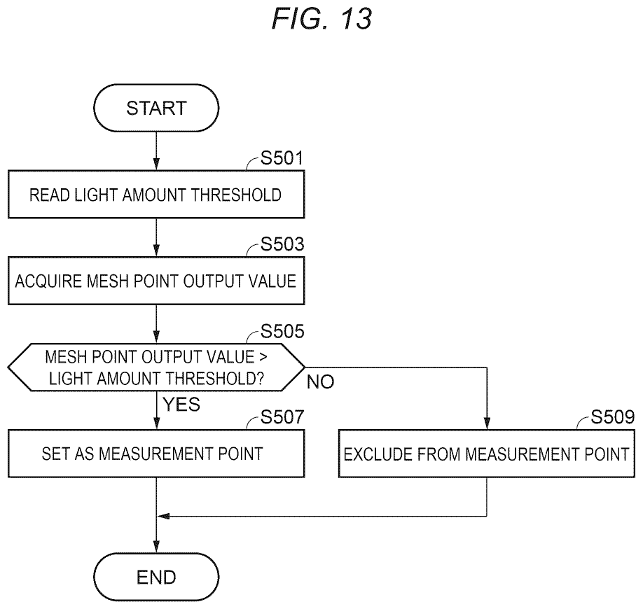

FIG. 13 shows an example of a control flow executed by the measurement apparatus 10. FIG. 13 shows a control flow for specifying a measurement point using time series data. FIG. 13 shows an example of the control flow executed in step S117 shown in FIG. 7. In FIG. 13, the measurement point is specified using a mesh point output value contained in the time series data. The measurement apparatus 10 acquires the pulse wave signal in step S119 shown in FIG. 7 using the time series data of the specified measurement point.

In step S501, the measurement apparatus 10 reads a light amount threshold. The light amount threshold is data used for comparison with the mesh point output value. For example, the light amount threshold indicates a lower limit value of the mesh point output value. The light amount threshold is set in advance and stored in the storage unit 41. The data processing unit 35 reads the light amount threshold from the storage unit 41.

After reading the light amount threshold, the measurement apparatus 10 acquires the mesh point output value in step S503. The mesh point output value corresponds to an example of feature point detection light amount. The data processing unit 35 acquires the mesh point output value contained in the time series data. The data processing unit 35 acquires the mesh point output value of each mesh point 131a for each piece of image data.

After acquiring the mesh point output value, the measurement apparatus 10 determines whether the mesh point output value is larger than the light amount threshold in step S505. When the mesh point output value is smaller than the light amount threshold, the detection accuracy of the pulse wave signal acquired using the mesh point output value is lowered. The data processing unit 35 acquires time series data including a mesh point output value larger than the light amount threshold.

The data processing unit 35 selects one mesh point 131a from the plurality of mesh points 131a. The data processing unit 35 compares the mesh point output value contained in the time series data of the one mesh point 131a with the light amount threshold. The data processing unit 35 sequentially selects all the mesh points 131a. The data processing unit 35 compares the mesh point output value contained in the time series data of the selected mesh point 131a with the light amount threshold for each mesh point 131a. When the data processing unit 35 determines that the mesh point output value is larger than the light amount threshold, the measurement apparatus 10 proceeds the processing to step S507 (step S505: YES). When the data processing unit 35 determines that the mesh point output value is smaller than the light amount threshold, the measurement apparatus 10 proceeds the processing to step S509 (step S505: NO).

In step S507, the measurement apparatus 10 sets, as a measurement point, the mesh point 131a whose mesh point output value contained in the time series data is larger than the light amount threshold. The measurement point is a tracking measurement point or an analysis measurement point. The data processing unit 35 selects one or more measurement points from the plurality of mesh points 131a by setting the mesh point 131a whose mesh point output value contained in the time series data is larger than the light amount threshold as a measurement point. The data processing unit 35 acquires time series data of the mesh point 131a set as the measurement point. The data processing unit 35 acquires a pulse wave signal in step S119 shown in FIG. 7 using the acquired time series data.

In step S509, the measurement apparatus 10 excludes the mesh point 131a whose mesh point output value contained in the time series data is smaller than the light amount threshold from a measurement point. The data processing unit 35 does not use the time series data of the mesh point 131a excluded from the measurement point for acquisition of the pulse wave signal.

The data processing unit 35 preferably acquires the mesh point output value of each of the plurality of mesh points 131a and selects an analysis measurement point using a plurality of the mesh point output values.

The measurement apparatus 10 selects the analysis measurement point using the mesh point output value, thereby improving the detection accuracy of the pulse wave signal.

In FIG. 13, the measurement point is selected by comparing the mesh point output value with the light amount threshold, but the present disclosure is not limited thereto. The light amount threshold may be an upper limit value. The light amount threshold may be a value for setting a light amount range including an upper limit value and a lower limit value. The data processing unit 35 may select a measurement point by comparing each mesh point output value with a statistic value. The statistic value is an example of the light amount threshold. The statistic value may be a value set based on a standard deviation, a variance, a median value, quartile points, or the like of the mesh point output value contained in one piece of time series data. The statistic value may be a value set based on a standard deviation, a variance, a median value, quartile points, or the like of mesh point output values contained in the selected time series data or all the time series data.

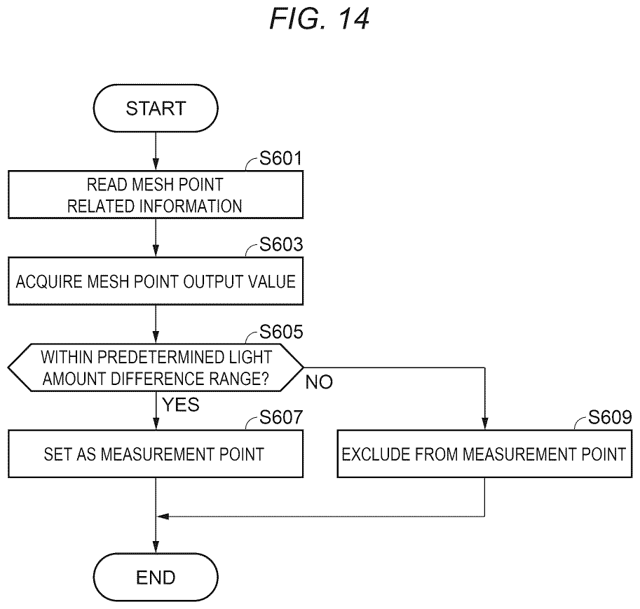

FIG. 14 shows an example of a control flow executed by the measurement apparatus 10. FIG. 14 shows a control flow for specifying a measurement point using the mesh point related information MT and the time series data. FIG. 14 shows an example of the control flow executed in step S117 shown in FIG. 7. In FIG. 14, the measurement point is specified using the mesh point related information MT and the mesh point output value contained in the time series data. The measurement apparatus 10 acquires the pulse wave signal in step S119 shown in FIG. 7 using the time series data of the specified measurement point.

In step S601, the measurement apparatus 10 reads the mesh point related information MT from the storage unit 41. For example, the data processing unit 35 reads the mesh point related information MT shown in FIG. 6.

After reading the mesh point related information MT, the measurement apparatus 10 acquires a mesh point output value in step S603. The data processing unit 35 acquires the mesh point output value contained in the time series data. The data processing unit 35 acquires the mesh point output value of each mesh point 131a for each piece of image data.

After acquiring the mesh point output value, the measurement apparatus 10 determines whether the mesh point output value of the adjacent mesh point 131a is within a predetermined light amount difference range in step S605. The data processing unit 35 selects one mesh point 131a from the plurality of mesh points 131a. The data processing unit 35 acquires a mesh point output value of the one mesh point 131a. The data processing unit 35 specifies the adjacent mesh point 131a adjacent to the one mesh point 131a using the mesh point related information MT. The data processing unit 35 acquires a mesh point output value of the adjacent mesh point 131a. The data processing unit 35 calculates a difference value between the mesh point output value of the one mesh point 131a and the mesh point output value of the adjacent mesh point 131a. The data processing unit 35 determines whether the difference value is within the predetermined light amount difference range. When the difference value is larger than the predetermined light amount difference, the data processing unit 35 determines that the mesh point output value of the one mesh point 131a is an abnormal value. The data processing unit 35 can improve the detection accuracy of the pulse wave signal by excluding the mesh point output value that may be an abnormal value.

When the data processing unit 35 determines that the difference value is within the predetermined light amount difference range, the measurement apparatus 10 proceeds the processing to step S607 (step S605: YES). When the data processing unit 35 determines that the difference value is not within the predetermined light amount difference range, the measurement apparatus 10 proceeds the processing to step S609 (step S605: NO).

In step S607, the measurement apparatus 10 sets the mesh point 131a whose difference value is within the predetermined light amount difference range as a measurement point. The measurement point is a tracking measurement point or an analysis measurement point. The data processing unit 35 selects one or more measurement points from the plurality of mesh points 131a by setting the mesh point 131a whose difference value is within the predetermined light amount difference range as a measurement point. The data processing unit 35 acquires time series data of the mesh point 131a set as the measurement point. The data processing unit 35 acquires a pulse wave signal in step S119 shown in FIG. 7 using the acquired time series data.

In step S609, the measurement apparatus 10 excludes the mesh point 131a whose difference value is not within the predetermined light amount difference range from the measurement point. The data processing unit 35 does not use the time series data of the mesh point 131a excluded from the measurement point for acquisition of the pulse wave signal.

In FIG. 14, the measurement point is selected using the difference value, but the present disclosure is not limited thereto. For example, the data processing unit 35 calculates an inter-mesh-point distance between adjacent mesh points 131a using mesh point coordinates. The data processing unit 35 calculates a light amount displacement amount using the inter-mesh-point distance and the mesh point output values of the two adjacent mesh points 131a. The data processing unit 35 may select a measurement point using the light amount displacement amount and a predetermined light amount displacement amount threshold.

What is claimed is:

1. A biometric information acquisition apparatus comprising:
an imaging unit configured to image a living body and generate a plurality of pieces of frame image data;
a face recognition unit configured to identify a face image contained in the frame image data and specify a plurality of feature points in the face image; and
a detection unit configured to detect a pulse wave signal of the living body, wherein
the detection unit is configured to
select a tracking feature point from the plurality of feature points, and track the tracking feature point contained in each of the plurality of pieces of frame image data, and
acquire the pulse wave signal based on a tracking feature point detection light amount of the tracking feature point in each of the plurality of pieces of frame image data.

2. The biometric information acquisition apparatus according to claim 1, wherein
the detection unit is configured to
select a plurality of measurement feature points including the tracking feature point from the plurality of feature points, and track the measurement feature points contained in each of the plurality of pieces of frame image data, and
calculate a measurement feature point detection light amount of the measurement feature points of each of the plurality of pieces of frame image data, and acquire the pulse wave signal using a plurality of the measurement feature point detection light amounts.

3. The biometric information acquisition apparatus according to claim 2, wherein
the detection unit is configured to acquire coupling information related to the feature points adjacent to each of the plurality of feature points.

4. The biometric information acquisition apparatus according to claim 2, wherein
the detection unit is configured to
acquire a feature point detection light amount of each of the plurality of feature points, and
select the measurement feature point from the plurality of feature points using a plurality of the feature point detection light amounts.

5. The biometric information acquisition apparatus according to claim 2, wherein
the detection unit is configured to
specify coordinate information of each of the plurality of feature points, and
select the measurement feature point based on the coordinate information.

6. The biometric information acquisition apparatus according to claim 3, wherein
the detection unit is configured to select the measurement feature point using the coupling information.

7. A non-transitory computer-readable storage medium storing a biometric information acquisition program for causing a computer, which is coupled to an imaging unit that images a living body and generates a plurality of pieces of frame image data, to execute the following processing of:
identifying a face image contained in the frame image data;
specifying a plurality of feature points in the face image;
selecting a tracking feature point included in the plurality of feature points;
tracking the tracking feature point contained in each of the plurality of pieces of frame image data; and
acquiring a pulse wave signal based on a tracking feature point detection light amount of the tracking feature point in each of the plurality of pieces of frame image data.
